(19) United States
(12) Patent Application Publication
(10) Pub. No.: US 2019/0076371 A1
(43) Pub. Date: Mar. 14, 2019

(54) MAGNETIC FIELD-CONTROLLED MICROROBOT FOR CARRYING AND DELIVERING TARGETED CELLS

(71) Applicant: City University of Hong Kong, Kowloon (HK)

(72) Inventors: Dong Sum, Hong Kong (HK); Junyang Li, Hong Kong (HK)

(21) Appl. No.: 15/702,462

(22) Filed: Sep. 12, 2017

(51) Int. Cl. A61K 9/50; A61B 6/12; A61K 9/00; A61F 2/02; A61N 2/06; A61L 27/04; A61L 27/54; A61L 27/56; A61L 27/58; A61K 47/50

(52) U.S. Cl. CPC A61K 9/5094; A61F 2230/0076; A61K 9/0009; A61F 2/02; A61N 2/06; A61L 27/04; A61L 27/54; A61L 27/56; A61L 27/58; A61K 47/50; A61F 2210/0004; A61F 2210/009; A61F 2210/0085; A61F 2310/00023; A61F 2310/00017; A61F 2310/00155; A61F 2310/00473; A61F 2310/00047; A61B 6/12

(57) ABSTRACT

Magnetically driven biocompatible microrobots comprising a porous body having a magnetic layer and a biocompatible layer configured to carry and deliver cells to desired sites are described. Embodiments of microrobots are configured with enhanced cell-loading ability, such as by including a plurality of burr members disposed upon the porous body for configuring the microrobot for enhanced cell-loading. The magnetic layer of embodiments may be provided on some portion or all of a surface of the microrobot for configuring the microrobot to be controlled with an external magnetic field. The biocompatible layer of embodiments may be provided on some portion or all of a surface of the microrobot, possibly coating some or all of the aforementioned magnetic layer, for configuring the microrobot for improved biostability and biocompatibility.

### TECHNICAL FIELD

This invention relates generally to the production and use of microrobot structures and, more particularly, to magnetically driven microrobots configured to carry and deliver cells to desired sites.

### BACKGROUND OF THE INVENTION

Microrobots have been the subject of recent attention in light of their potential to revolutionize many aspects of medicine and their potential to enable new procedures never before possible. Microrobots might, for example, be used for the localized delivery of chemical and biological substances (e.g., pharmacotherapy agents, living tissue, etc.), to remove material (e.g., neoplastic tissue, osteophytes, etc.) by mechanical means, to act as simple static structures (e.g., stents, occlusions, implants, etc.), or to transmit information from a location within the body (e.g., a location from which it would otherwise be very difficult to obtain information).

Various configurations of microrobots have been developed for use in minimally invasive medical techniques which make some existing therapeutic and diagnostic procedures less invasive. However, the minimally invasive surgery used to introduce such microrobots into the patient's body is nevertheless invasive and requires an incision of some size with the aid of a mechanical instrument. Such open surgery may lead to infection and injury during the insertion.

Moreover, existing microrobots configured for carrying living tissue in the form of individual cells typically have a very limited cell load capacity and/or provide very limited transport ability. For example, S. Tottori, L. Zhang, F. Qiu, K. K. Krawczyk, A. Franco-Obregón, B. J. Nelson, “Magnetic helical micromachines: fabrication, controlled swimming, and cargo transport,” Advanced materials 24, 811-816 (2012) and S. Tottori, L. Zhang, F. Qiu, K. K. Krawczyk, A. Franco-Obregón, B. J. Nelson, “Magnetic helical micromachines: fabrication, controlled swimming, and cargo transport,” Advanced materials 24, 811-816 (2012), the disclosures of which are hereby incorporated herein by reference, discuss helical magnetic swimming micromachines with a microholder and a U-shaped magnetic microrobot, respectively, each of which can only transport a single cell with very limited transport ability. S. Kim, F. Qiu, S. Kim, A. Ghanbari, C. Moon, L. Zhang, B. J. Nelson, H. Choi, “Fabrication and characterization of magnetic microrobots for three-dimensional cell culture and targeted transportation,” Advanced Materials 25, 5863-5868 (2013), the disclosure of which is hereby incorporated herein by reference, discusses microrobots having a structure supporting very limited cell load capacity.

Patent documents US20090098183A1, WO2005095581A1, WO1988003785A1, and WO2012149358A1, the disclosures of which are hereby incorporated herein by reference, describe a scaffold-like 3D structure for implanting to repair damage tissue or organ that provides very limited cell load capacity and that does not provide magnetic actuation ability and cannot be used for magnetic driven cell transportation in body. Patent documents U.S. Pat. No. 8,900,293 and U.S. Pat. No. 7,846,201, the disclosures of which are hereby incorporated herein by reference, discuss paramagnetic particles associated with a therapeutic agent, drug, and/or a cell to be delivered to a targeted location, wherein the paramagnetic particles provide a relatively low magnetization under applicable external magnetic fields, thus resulting in very limited transport ability. Patent document US20130017229, the disclosure of which is hereby incorporated herein by reference, discusses a magnetic porous scaffold in centimeter size, which can act as a depot of various cells, and the cell release can be controlled by external magnetic fields, wherein the porous scaffold structure is difficult to be used in vivo and thus is impractical for providing cell transport ability with respect to living tissue applications. Patent document US20110270434, the disclosure of which is incorporated herein by reference, discusses magnetic nanostructured propellers to detect biomolecules that work for the controlled transportation of molecules and the delivery of microscopic and nanoscale objects to targeted cell, wherein such nanostructured propellers cannot be used for carrying and delivering cells. Patent document US20120253102, the disclosure of which is hereby incorporated herein by reference, discusses superparamagnetic microspheres label cells to enhance cell retention, engraftment, and functional benefit, wherein the microspheres are not designed to transport the cells in vivo. Patent documents US20140302110 and US20150351897A1, the disclosures of which are incorporated herein by reference, describe magnetic bio-scaffold to transport multiple cells, however the cell load capacity remains very limited.

### BRIEF SUMMARY OF THE INVENTION

The present invention is directed to systems and methods which provide magnetically driven biocompatible microrobots comprising a porous body having a magnetic layer and a biocompatible layer configured to carry and deliver cells to desired sites. The porous body of embodiments of a microrobot herein may comprise a polymeric, ceramic, or nanofiber three-dimensional structure. The porous structure of the body may, for example, be configured to mimic the extracellular matrix, in which nutrients can be easily supplied for tissue vascularization to yield functional tissues and organs. The pores in the porous body of embodiments may be sized in correspondence with the type of cells to be carried by the microrobot. The magnetic layer of embodiments may be provided on some portion or all of a surface of the microrobot for configuring the microrobot to be controlled with an external magnetic field, such as to enable the magnetic microrobot to be positioned at a target site without the use of surgery or mechanical instrument. The biocompatible layer of embodiments may be provided on some portion or all of a surface of the microrobot, possibly coating some or all of the aforementioned magnetic layer, for configuring the microrobot for improved biostability and biocompatibility. A microrobot coated with magnetic materials and biocompatible materials according to embodiments herein is thus configured to allow magnetic control of the microrobot and to facilitate cell cultivation on it.

Embodiments of microrobots of the present invention are configured with enhanced cell-loading ability. For example, such embodiments may include a plurality of burr members disposed upon the porous body for configuring the microrobot for enhanced cell-loading. The burr members may, for example, comprise relatively thin protrusions extending generally orthogonally from the surface of the microrobot porous body, wherein the interspacing of the burr members may correspond with the type of cells to be carried by the microrobot. Cells to be carried and/or delivered to desired sites may, for example, each be cultured between adjacent burr members disposed on the outside of a microrobot of embodiments herein. The burr member structure of embodiments herein allows more cells to adhere onto the microrobot, facilitating a higher cell-loading ability for the microrobot.

In operation of embodiments herein, a magnetically driven biocompatible microrobot is used to carry and deliver targeted cells to a desired site, without the use of surgical techniques to deliver the microrobot directly to the desired site. The carried cells can be spontaneously released to the surrounding tissues from a microrobot configured in accordance with the concepts herein when the microrobot arrives at the desired site; as confirmed by in vitro and in vivo experiments. For example, the porous three-dimensional structure of microrobots of embodiments herein, particularly when adapted to include burr members in accordance with concepts herein, have been proven to effectively release cells spontaneously when disposed at a desired site.

It should be appreciated that microrobots configured in accordance with the foregoing embodiments present a great potential for future applications in tissue repair and regeneration in precision medicine. Embodiments of a magnetically driven biocompatible microrobot may be utilized in facilitating new approaches to organ regeneration.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWING

For a more complete understanding of the present invention, reference is now made to the following descriptions taken in conjunction with the accompanying drawing, in which:

FIGS. 1A and 1B show an exemplary embodiment of a magnetically driven biocompatible microrobot configured in accordance with embodiments of the present invention;

FIG. 2 shows a high level flow diagram of operation in accordance with a fabrication technique of embodiments of the present invention;

FIG. 3 shows an implementation of the flow of FIG. 2 illustrating the construction of a magnetically driven biocompatible microrobot of embodiments of the present invention;

FIG. 4A is a scantling electron microscopic (SEM) image of a magnetically driven biocompatible microrobot constructed in accordance with the flow of FIG. 2 using an implementation as shown in FIG. 3;

FIGS. 4B and 4C shows the magnetically driven biocompatible microrobot of FIG. 4A having cells cultured thereon;

FIG. 5A shows a comparison of magnetically driven microrobots with and without burr members thereon;

FIG. 5B shows the magnetically driven biocompatible microrobot of FIG. A with burr members having cells cultured thereon;

FIG. 5C shows the magnetically driven biocompatible microrobot of FIG. 5A without burr members having cells cultured thereon;

FIGS. 6A-6C show epochs of a control experiment of single cell-cultured magnetically driven biocompatible microrobots in vitro according to an exemplary embodiment of the present invention;

FIGS. 7A-7C show epochs of a control experiment of single cell-cultured magnetically driven biocompatible microrobots in vivo according to an exemplary embodiment of the present invention;

FIGS. 8A-8D show release of MC3T3-E1 cells carried by a cell-cultured magnetically driven biocompatible microrobot of an embodiment of the present invention;

FIGS. 9A, 9B, 10A, 10B, 11A, and 11B show release of mesenchymal stem cells (MSCs) labeled with a fluorescent reagent from a cell-cultured magnetically driven biocompatible microrobot of an embodiment of the present invention;

FIGS. 12A-12D show release of cells from a swarm of cell-cultured magnetically driven biocompatible microrobots of an embodiment of the present invention injected subcutaneously into a nude mouse; and

FIGS. 13A-13D show a control group of a similarly sized swarm of magnetically driven biocompatible microrobots without the cells carried by the microrobots of FIGS. 12A-12D injected subcutaneously into the nude mouse.

### DETAILED DESCRIPTION OF THE INVENTION

The concepts described herein provide for the design and fabrication of magnetically driven biocompatible microrobots, such as may be utilized as targeted cells carrying microrobots. For example, functional cells, such as MC3TE-E1 and mesenchymal stem cells (MSCs), can be carried and transferred by the magnetically driven biocompatible microrobots of embodiments of the invention. In operation according to some embodiments, MSCs can be delivered and released because MSCs can potentially differentiate into various cell types of the host tissues, such as adipocytes, chondrocytes, and myocytes. Irrespective of the particular cells for which a magnetically driven biocompatible microrobot may be configured to carry, the microrobots of embodiments of the invention are configured for controlling the movement of microrobot using a magnetic field applied, whereby cells can be carried and released to a localized part of the human body.

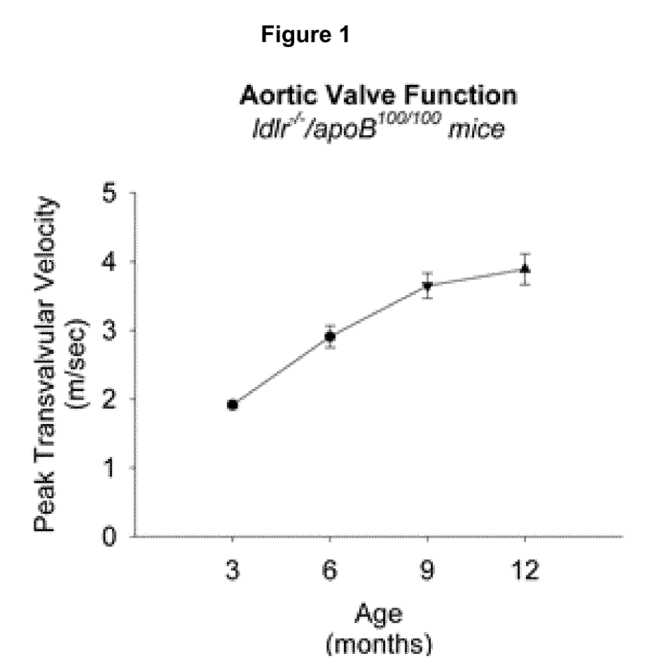

FIG. 1A shows an exemplary embodiment of a magnetically driven biocompatible microrobot configured in accordance with the concepts herein. Microrobot 100 of the illustrated embodiment comprises a three-dimensional structure including porous body 110 having a plurality of pores 111 disposed therein.

Porous body 110 of embodiments of the invention may, for example, comprise a three-dimensional structure in the form of cylinder, a hexahedron, an ellipsoid, a polyhedron, a circular cone, or a sphere, such as may be formed from one or more materials (e.g., polymer, ceramic, nanofiber, etc.). The size and shape of porous body 110, and correspondingly microrobot 100, may vary depending on the type of the cells to be carried and/or delivered, the physiology of the target delivery site, the morphological attributes of an expected path to the target delivery site, etc. In accordance with some embodiments herein, porous body 110 may comprise a three-dimensional structure having dimensions (e.g., diameter, length, width, and/or height) preferably in the range of 1-1000 micrometer, and more preferably in the range of 30-100 micrometer, because microrobots with the foregoing dimensions can readily move within the body such as in vessels, cerebral ventricles, and viscoelastic media and fluid of organs with the help of magnetic field. Moreover, porous body 110 of embodiments may comprise a sphere because a spherical structure may easily fuse with host tissue and thus facilitate cell transfer from the microrobot to the tissues. The porous structure of porous body 110 of the illustrated embodiment is configured to mimic the extracellular matrix to facilitate the supply of nutrients for tissue vascularization to yield functional tissues and organs. Pores 111 of porous body 110 of the illustrated embodiment are sized in correspondence with the type of cells to be carried by microrobot 100. For example, the diameter of the orifice of pores 111 in the outer surface of porous body 110 is sized in correspondence with an average circumference of the particular cells (e.g., the average interphase length or circumference of targeted cells) to be carried by microrobot 100.

It should be appreciated that the porous structure of porous body 110 may itself be utilized to carry cells. For example, cells to be carried and/or delivered to desired sites may each be cultured within an orifice of one of pores 111, thus being disposed between opposing edges of the pore orifice (e.g., through cell adhesion to biocompatible material of microrobot 100). However, microrobot 100 of the illustrated embodiment includes a plurality of burr members 120 utilized to carry cells.

Burr members 120 of embodiments comprise relatively thin protrusions extending generally orthogonally from the outer surface of porous body 110. Each burr member 120 may, for example, comprise a protuberance extending from the outer surface of porous body 110 in the form of a cylinder, a hexahedron, an ellipsoid, or a polyhedron. Burr members 120 of embodiments are adapted so as not to be so long as to injure tissues/organs as the microrobots move in the body and so as to not be so short as to discourage solid cell attachment thereto. Each burr member 120 may, for example, preferably he preferably in the range of 1-30 micrometers, and more preferably from 5-15 micrometers. Moreover, the distal end of burr members 120 of embodiments may be adapted so as to decrease the potential damage to the tissues and organs when these cell-cultured microrobots are delivered to the desired position in the body. For example, burr members 120 of the illustrated embodiment include ball terminal structures at the distal end thereof for minimizing the potential for damage to tissues by the microrobots. The cross section size of burr members 120 may preferably be in the range of 1-10 micrometers, and more preferably from 2-5 micrometers, according to some embodiments. Burr members 120 of the illustrated embodiment are provided with an interspacing corresponding with an average circumference of the particular cells (e.g., the average interphase length or circumference of targeted cells) to be carried by microrobot 100. For example, burr members 120 shown in FIG. 1A are disposed on the outer surface of porous body 110 at or near the edges of pores 111 such that the interspacing of pairs of burr members 120 corresponds to the diameter of pores 111, wherein the diameter of the orifice of pores 111 is sized in correspondence with the particular cells to be carried by microrobot 100.

Although the illustrated embodiment shows a configuration of microrobot 100 in which both the size of the orifice of pores 111 and the interspacing of burr members 120 correspond to a size of the particular cells to be carried by the microrobot, there is no limitation with respect to both the sizing of the pores and the spacing of the burr members corresponding to a size of cells to be carried. Embodiments of a magnetically driven biocompatible microrobot may, for example, comprise pores having a diameter sized to facilitate the supply of nutrients for tissue vascularization to yield functional tissues and organs without necessarily corresponding to a size of the particular cells to be carried by the microrobot, whereas the burr member interspacing may correspond to the size of the particular cells to be carried by the microrobot

Burr members 120 of the illustrated embodiment of microrobot 100 are disposed upon porous body 110 for configuring the microrobot for enhanced cell-loading. Cells to be carried and/or delivered to desired sites (e.g., cells 101 of FIG. 1B, showing microrobot 100 after cell seeding) may each be cultured between adjacent ones of burr members 120 (e.g., through cell adhesion to biocompatible material of microrobot 100) disposed on the outside of the microrobot. The open structure of the burr member configuration of such embodiments allows more cells (e.g., as compared to an embodiment utilizing the closed structure of the pore orifices to carry cells) to adhere onto the microrobot, facilitating a higher cell-loading ability for the microrobot.

It should be appreciated that, although the foregoing exemplary embodiment having a plurality of burr members has been described with respect to cells cultured between burr members, cells may additionally be cultured elsewhere in the microrobot structure. For example, cells may be cultured within an orifice of one of pores of the porous body of embodiments having a plurality of burr members. Moreover, it should be appreciated that the three-dimensional structure of microrobots of embodiments (e.g., mircrorobot 100 of the illustrated embodiment) herein facilitates three-dimensional cell cultures, such as is useful for sustaining the structural and functional complexities of the cells.

Microrobot 100 of the illustrated embodiment is a magnetically driven biocompatible microrobot configuration. Accordingly, microrobot 100 of embodiments comprises a magnetic layer and a biocompatible layer configured to carry and deliver cells to desired sites. The magnetic layer of embodiments may be provided on some portion or all of a surface of microrobot 100, such as to coat porous body 110 and/or burr members 120. The biocompatible layer of embodiments may likewise be provided on some portion or all of a surface of the microrobot, such as to coat porous body 110 and/or burr members 120, possibly coating some or all of the aforementioned magnetic layer.

The magnetic layer of microrobot 100 of embodiments is provided for configuring the microrobot to be controlled with an external magnetic field, such as to enable the magnetic microrobot to be positioned at a target site without the use of surgery or mechanical instrument. Accordingly, a type of magnetic layer material, sufficient amount of magnetic layer material, and/or a placement of magnetic layer material upon the microrobot structure may be selected according to embodiments to facilitate control of the microrobot. In accordance with embodiments of the invention, the magnetic layer material is provided so as to be thick enough to facilitate magnetism that is strong enough to control movement of the microrobot while being thin enough to accommodate a deposit time for the material that is conducive to efficient manufacturing processes. For example, the magnetic layer material of embodiments of microrobot 100 is preferably 50-300 nm thick, and more preferably 100-200 nm thick. Magnetic material of magnetic layer of microrobot 100 may comprise a metal having a suitable level of magnetism for facilitating control of the microrobot, and without significant corrosiveness (reactivity). The composition of the magnetic layer may, for example, include nickel (Ni), iron (Fe), cobalt (Co), and neodymium (Nd), or a combination thereof. A preferred embodiment of microrobot 100 comprises a magnetic layer formed from a metal composition containing Ni.

The biocompatible layer of microrobot 100 of embodiments is provided for configuring the microrobot for improved biostability and biocompatibility. Accordingly, a type of biocompatible layer material, a sufficient amount of biocompatible layer material, and/or a placement of biocompatible layer material upon the microrobot structure may be selected according to embodiments to facilitate cell cultivation and adhesion on the microrobot. In accordance with embodiments of the invention, the biocompatible layer material provided so as to be thick enough that suitable biocompatibility is facilitated while being thin enough to accommodate a deposit time for the material that is conducive to efficient manufacturing processes. For example, the biocompatible layer of embodiments of microrobot 100 is preferably 10-50 nm in thick. Biocompatible material of magnetic layer of microrobot 100 may comprise a metal exhibiting a high biocompatibility for facilitating microrobot biostability and biocompatibility. The composition of the biocompatible layer may, for example, include titanium (Ti), medical stainless steel, alumina (Al₂O₃), and gold (Au), or a combination thereof. A preferred embodiment of microrobot 100 comprises a biocompatible layer formed from a metal composition containing Ti.

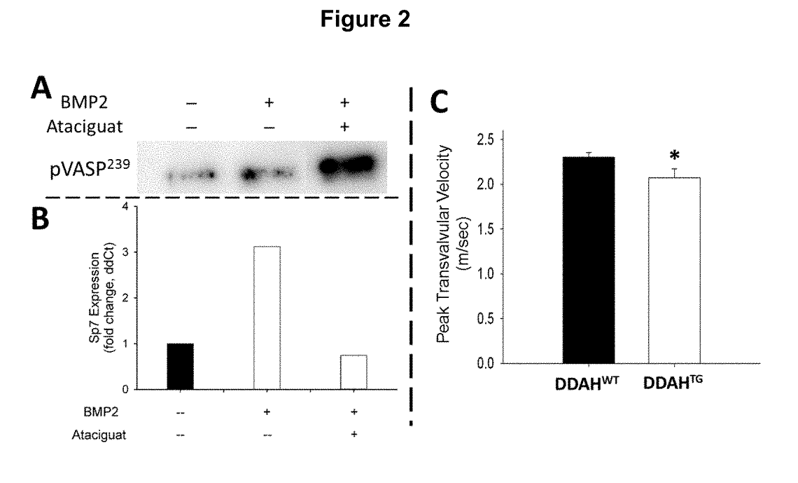

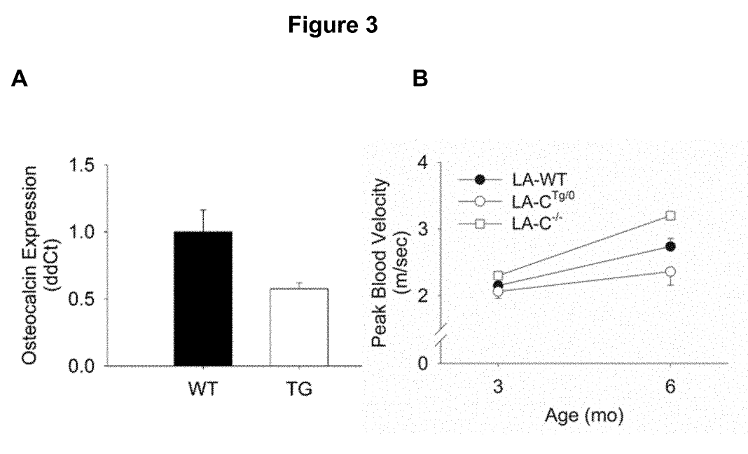

FIGS. 2 and 3 illustrate a fabrication technique for providing magnetically driven biocompatible microrobots of embodiments of the present invention, such as microrobot 100 of FIGS. 1A and 1B. In particular, FIG. 2 shows a high level flow diagram of operation in accordance with a fabrication technique of embodiments and FIG. 3 illustrates an implementation of the flow of FIG. 2.

In operation according to the illustrated embodiment of flow 200 shown in FIG. 2, porous microrobot structure (e.g., comprising porous body 110, and as may comprise burr members 120) for the microrobot is first formed at block 210. For example, three-dimensional porous microrobot structure may be formed by lithography. Accordingly, in embodiments of the invention the porous microrobot structure may be formed from a photocurable polymer, such as SU-8 polymer, IP-L, IP-G, and a combination thereof (e.g., in a preferred embodiment, SU-8 is used to form the porous microrobot structure of microrobot 100). For example, a volume of such a photocurable polymer may be exposed to light (e.g., a particular wavelength and/or intensity of light to which the photocurable polymer is reactive), such as using a controllable, focused light source (e.g., LASER), as represented by block 211 of FIG. 2 and step 311 of FIGURE. 3. The photocurable polymer of this example is cured upon exposure to the light, and thus the desired porous microrobot structure is defined (e.g., “written”) using the controlled exposure to light. Thereafter, the porous microrobot structure may be developed to stabilize the photocurable polymer forming the porous microrobot structure and/or remove unexposed photocurable polymer, as represented by block 212 of FIG. 2 and step 312 of FIG. 3. For example, the volume of photocurable polymer may be drained to reveal the porous microrobot structure formed from the photocurable polymer that was exposed to light and/or the photocurable polymer that was exposed to light and forming the porous microrobot structure may be exposed to one or more agents (e.g., chemical stabilizer, heat, particular wavelength of light, etc.) for stabilizing the photocurable polymer forming the porous microrobot structure,

It should be appreciated that, although the foregoing example of forming a porous microrobot structure described with respect to block 210 uses a lithographic technique, embodiments of the present invention may use additional or alternative techniques in forming porous microrobot structures of a magnetically driven biocompatible microrobot. For example, operation to form porous microrobot structure in accordance with block 210 of embodiments of the invention may use epitaxy, molecular deposition, microextrusion, particulate leaching, emulsion freeze-drying, fused deposition modeling (FDM), etc.

Having formed the underlying porous microrobot structure for the magnetically driven biocompatible microrobot, flow 200 of the illustrated embodiment proceeds to block 220 wherein a magnetic layer material is disposed upon a surface of the porous microrobot structure. For example, a deposition technique, such as electron beam deposition, dipping, electroplating, sputtering, chemical vapor deposition, direct metal deposition, etc., may be used to deliver a magnetic layer material to an outer surface of the porous microrobot structure (as represented by step 320 of FIG. 3) and form a magnetic layer suitable for controlling the microrobot with an external magnetic field.

After forming the magnetic layer, flow 200 of the illustrated embodiment proceeds to block 230 wherein a biocompatible layer material is disposed upon a surface of the porous microrobot structure. For example, a deposition technique, such as electron beam deposition, dipping, electroplating, sputtering, chemical vapor deposition, direct metal deposition, etc., may be used to deliver a biocompatible layer material to an outer surface of the porous microrobot structure (as represented by step 330 of FIG. 3) and form a biocompatible layer suitable for providing biostability and biocompatibility with respect to the microrobot.

Having the magnetic layer and biocompatible layer formed upon the porous microrobot structure, a magnetically driven biocompatible microrobot of embodiments is provided after completion of block 230 of flow 200. The resulting magnetically driven biocompatible microrobot may, for example, be utilized as a targeted cells carrying microrobot. Accordingly, the microrobot of embodiments may be introduced to a cell culture in order for a plurality of cells to be cultured in the structure of the microrobot (e.g., cells cultured between adjacent ones of the burr members of an embodiment having a plurality of burr members, cells cultured within an orifice of one of pores of an embodiment without burr members, etc.), as represented by step 340 of FIG. 3.

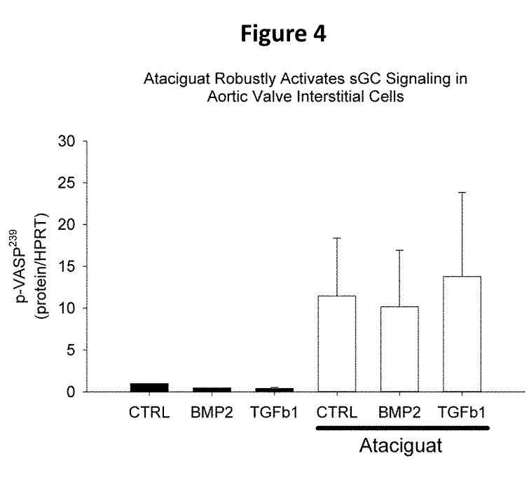

FIGS. 4A-4C show scanning electron microscope (SEM) images of an embodiment of a magnetically driven biocompatible microrobot configured as described above with respect to microrobot 100. Accordingly, FIG. 4A shows a microrobot with a porous body having a plurality of burr members. The distance between pairs of burr members of the microrobot of FIG. 4A may, for example, be approximately 10-20 μm, allowing a capacity of a human cell. However, it should be appreciated that the pore size can be adjusted, such as using 3D laser lithography as described above, to suit different sizes of cells.

Magnetically driven biocompatible microrobots configured as shown in FIG. 4A may be fabricated in accordance with flow 200 of FIG. 2 in an implementation as illustrated in FIG. 4. For example, in forming the porous microrobot structure through a writing process, a 100 μm-thick SU-8 layer (MicroChem, USA) may be spin-coated onto a cleaned glass wafer under a suitable spin speed. The spin-coated substrate may then be pre-baked at 65° C. for 10 min and then at 95° C. for 30 min, then subsequently cooled to room temperature. The porous microrobot structure may be written into the Su-8 photoresist by using a commercial two-photon direct writing system (Nanoscribe GmbH, Germany) with an oil immersion objective of 100× (NA=1.4 from Zeiss; where NA denotes numerical aperture). In a development process, a post-bake may be conducted at 65° C. for 1 min and then baked at 95° C. for 10 mm. Thereafter, propylene glycol methyl ether acetate provided by Sigma Chemical Company (USA) may be used to develop the written porous microrobot structure and remove unpolymerized SU-8 for 20 minutes. The resulting porous microrobot structures may then be coated with Ni (e.g., 99.99% pure) for magnetic actuation and Ti (e.g., 99.99% pure) for biocompatibility (150 nm Ni and 20 nm Ti) by using Quorum Q150TS Dual Target Sputtering System (Quorum Technologies Inc., Canada).

As shown in FIGS. 4B and 4C, cells can be cultured onto the microrobot of FIG. 4A. Specifically, in the example of FIGS. 4B and 4C, MC3T3-E1 cells (FIG. 4B) and MSCs (FIG. 4C) are cultured into microrobots configured as shown in FIG. 4A after 12 hours at a concentration of 1×10⁶ cells/mL. In culturing the cells onto the microrobots, the fibroblasts MC3T3-E1 cells and MSCs may be maintained separately in DMEM supplemented with 10% fetal bovine serum, 100 U/mL penicillin, and 100 U/mL streptomycin at 37° C. in a humidified atmosphere of 5% CO₂. These two cell types may be subsequently trypsinized and resuspended at a concentration of 1×10⁶ cells/mL for cell seeding. The microrobots may be coated with 10 μg/mL of Poly-L-lysine (PLL; a positively charged. synthetic amino acid chain widely used as a coating material to enhance cell attachment because cell surfaces are always negatively charged) followed by sterilization by ultraviolet irradiation for 30 minutes. Thereafter, MC3T3 cells and MSCs may be seeded into culture dishes containing the respective microrobots and each dish with cells and microrobots stored in an incubator at 37° C. under a humidified atmosphere of 5% CO₂ for 12 hours. As can be seen in FIGS. 4B and 4C, in these examples, each cell is located between two adjacent burr members on the surface of the microrobot.

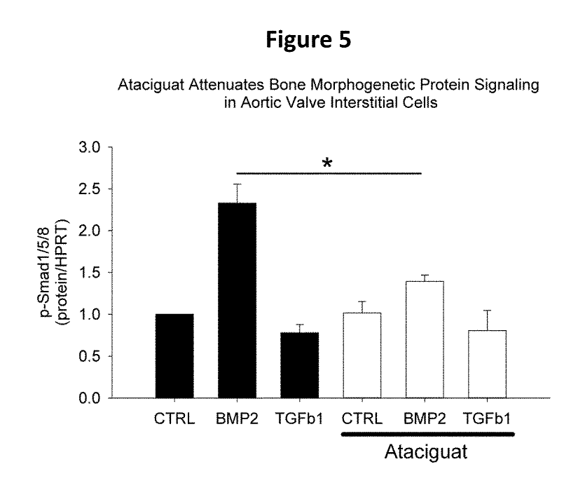

FIGS. 5A-5C show SEM images comparing magnetically driven biocompatible microrobots configured with and without burr members. In particular, FIG. 5A shows a magnetically driven biocompatible microrobot having a plurality of burr members on the left and a magnetically driven biocompatible microrobot having no burr members on the right. In the images of FIGS. 5E and 5C, cells have been cultured onto the two microrobot configurations. It can be seen by comparing the microrobot of FIG. 5B having a plurality of burr members and the microrobot of FIG. 5C having no burr members that the embodiment having the burr members allowed more cells to adhere onto the microrobot, indicating that the microrobot with burr members exhibits a higher cell-loading ability than that without burr members.

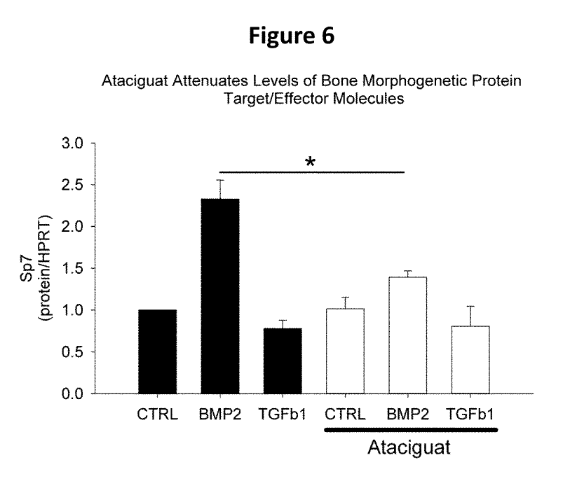

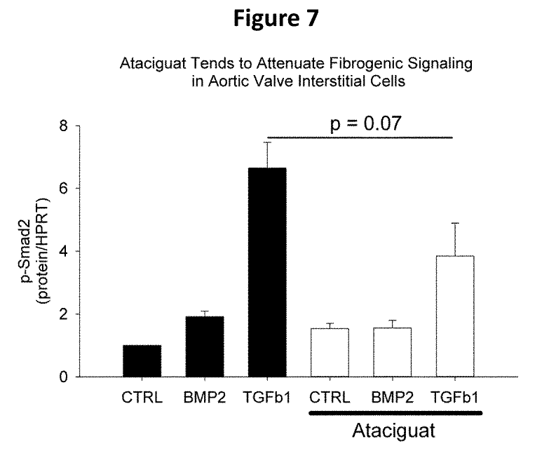

FIGS. 6A-6C and 7A-7C show epochs of control experiments of single cell-cultured magnetically driven biocompatible microrobots in vitro (FIGS. 6A-6C) and in vivo (FIGS. 7A-7C) according to an exemplary embodiment of the present invention. The magnetic manipulation system utilized for providing the magnetic field for driving the microrobots in the control experiments of FIGS. 6A-6C and 7A-7C comprised fixed DT4E-core identical electromagnetic coils (a soft magnetic material). In operation, after loading induction currents into coils, a magnetic field with field gradient can be generated. During the experiment, a container with cell-cultured microrobot is placed in the center of the workspace and controlled by electromagnetic coils. Accordingly, the cell-cultured microrobots of embodiments herein can be controlled to reach a desired position in vitro and in vivo, driven by such an electromagnetic manipulation system.

The control experiment of FIGS. 6A-6C show that cell-cultured magnetically driven biocompatible microrobots configured according to the concepts herein may be precisely controlled by a magnetic field according to the present invention. In particular, FIGS. 6A-6C, show a cell-cultured magnetically driven biocompatible microrobot moving along a triangular path through three control positions, a, b, and c. In the control experiment of FIGS. 6A-6C, the aforementioned electromagnetic manipulation system was used to provide in vitro transportation of a MC3T3-E1 cell-cultured microrobot along a desired triangular path to reach a targeted position by changing the induced current loaded to each electromagnetic coil. FIGS. 7A-7C show that cell-cultured magnetically driven biocompatible microrobot configured according to the concepts herein may be controlled by a magnetic field to directly carry cells in the animal model, such as nude mice or zebrafish. In particular, the control experiment of FIGS. 7A-7C uses a zebrafish as the animal model because of its genetic similarity to humans, it is transparent, and provides a relatively large yolk for microrobot transportation. In the control experiment of FIGS. 7A-7C, the aforementioned electromagnetic manipulation system was used to provide in vivo transport of a MSCs-cultured microrobot in the transparent yolk of zebrafish embryos, which allow easy visualization and monitoring of the in vivo movement of the microrobot on line, by changing the induced current loaded to each electromagnetic coil. FIGS. 7A-7C show the movement of a cell-cultured magnetically driven biocompatible microrobot carrying MSCs along a desired path at different time instants in the yolk of a zebrafish embryo. The transparent yolk of the zebrafish embryos allows easily visualization and monitoring of the real-time movement of the magnetically driven biocompatible microrobot in vivo. As can be appreciated from the control experiments of FIGS. 6A-6C and 7A-7C, magnetically driven biocompatible microrobots of embodiments of the present invention need not to be inserted directly into a target site by surgery and with the aid of mechanical instrument, but instead may be positioned under the control of an external magnetic field without risk of infection and injury,

FIGS. 8A-8D show phase-contrast images of MC3T3-E1 cells carried by a cell-cultured magnetically driven biocompatible microrobot of an embodiment in contact with a pure glass substrate. As can be seen in FIG. 8B, after 1 day of cell culture, six MC3T3-E1 cells were released from the microrobot and firmly attached to the glass substrate. As can be seen in FIG. 8C, after 2 days of cell culture, the released MC3T3-E1 cells differentiated and eleven were found on the glass substrate. As can be seen in FIG. 8D, after 3 days of cell culture, twenty-five MC3T3-E1 cells proliferated over the substrate. These results confirm successful cell delivery from the microrobot and cell growth on the substrate in vitro.

FIGS. 9A, 9B, 10A, 10B, 11A, and 11B show images of a cell-cultured magnetically driven biocompatible microrobot of an embodiment carrying cultured MSCs labeled with a fluorescent reagent placed onto a glass substrate with pre-cultured C2C12 cells to simulate a microtissue. In particular, FIGS. 9A and 9B show bright field images of the microrobot at two epochs, FIGS. 10A and 10B show fluorescence images of the microrobot at the two epochs, and FIGS. 11A and 11B show combined bright field and fluorescence images of the microrobot at the two epochs. As can be seen from FIGS. 9B, 10B, and 11B, after 7 days of cultivation, the labeled MSCs proliferated over the substrate. These results again confirm that the cells carried by the microrobot can be successfully transferred onto a desired site in vitro.

FIGS. 12A-12D show images of a swarm of cell-cultured magnetically driven biocompatible microrobots injected subcutaneously into the left dorsum of a nude mouse. FIGS. 13A-13D show images of a similarly sized swarm of magnetically driven biocompatible microrobots without the cells carried by the microrobots of FIGS. 12A-12D injected subcutaneously into the right dorsum of the nude mouse as a control. The swarm of microrobots of FIGS. 12A-12D carried Hela GFP⁺ cells dispersed in PBS and Matrigel to illustrate the in vivo releasing capacity of the microrobots because such tumorigenic cells facilitate the growth of tumors that can be easily detected in weeks. As can be seen in FIG. 12D, after 4 weeks of cultivation, an area with increased fluorescence intensity is clearly observed at the left dorsum of the mouse. However, as can be seen in FIG. 13D, after 4 weeks of cultivation, no tumor is found in the right dorsum of the mouse after injection with microrobots without Hela cells. This indicates that the tumor generates due to the Hela cancer cells carried by the injected swarm of microrobots.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

1. A microrobot configured to be magnetically driven and biocompatible, the microrobot comprising:
a porous body having a three-dimensional structures;
a plurality of burr members disposed on the porous body, wherein burr members of the plurality of burr members extend orthogonally from an outer surface of the porous body and are configured for carrying cells to desired sites by the microrobot between adjacent burr members of the plurality of burr members;
a magnetic layer coating at least a portion of the porous body or at least a portion of the burr members; and
a biocompatible layer coating at least a portion of the burr members for cell adhesion between adjacent burr members of the plurality of burr members.

2. The microrobot of claim 1, wherein the porous body comprises:
a photocurable polymer.

3. The microrobot of claim 1, wherein the three-dimensional structure comprises a structure selected from the group consisting of:
a cylinder;
a hexahedron;
an ellipsoid;
a polyhedron;
a circular cone; and
a sphere.

4. The microrobot of claim 1, wherein pores of the porous body are sized in correspondence with a type of cells to be carried by the microrobot.

5. The microrobot of claim 1, wherein the porous body is configured to mimic an extracellular matrix in which nutrients are supplied for tissue vascularization to yield functional tissues.

6. The microrobot of claim 1, wherein the magnetic layer comprises a metal selected from the group consisting of:
nickel (Ni);
iron (Fe);
cobalt (Co);
neodymium (Nd); and
combinations thereof.

7. The microrobot of claim 1, wherein the biocompatible layer fully covers the magnetic layer.

8. The microrobot of claim 1, wherein the biocompatible layer comprises a metal selected from the group consisting of:
titanium (Ti);
medical stainless steel;
alumina (Al₂0₃):
gold (Au); and
combinations thereof.

9. The microrobot of claim 1, wherein burr members of the plurality of burr members include ball terminal structures at a distal end thereof configured for minimizing potential for damage to tissues by the microrobot.

10. The microrobot of claim 1, wherein pores of the porous body are sized to facilitate supply of nutrients for tissue vascularization, and wherein adjacent burr members of the plurality of burr members have an interspacing corresponding with a type of cells to be carried by the microrobot.

11. The microrobot of claim 1, wherein each burr member of the plurality of burr members comprises:
a protuberance extending from a surface of the porous body, wherein the protuberance is formed in a shape selected from the group consisting of a cylinder, a hexahedron, an ellipsoid, and a polyhedron.

12. The microrobot of claim 1, wherein the plurality of burr members are configured for culturing cells between adjacent burr members of the plurality of burr members.

13. A system for delivery of cells to a desired biological site, the system comprising:
a plurality of microrobots configured to be magnetically driven and biocompatible, each microrobot of the plurality of microrobots including a porous body having a three-dimensional structure with a plurality of burr members extending orthogonally from an outer surface of the porous body, a magnetic layer coating at least a portion of the porous body and configured to enable the microrobot to be positioned at a target site using a magnetic field, and a biocompatible layer coating at least a portion of the burr members for cell adhesion between adjacent burr members of the plurality of burr members and configured for microrobot biostability and biocompatibility, wherein the plurality of burr members are configured for carrying cells to desired sites by the microrobot between adjacent burr members of the plurality of members.

14. The system of claim 13, wherein the porous body of microrobots of the plurality of micro comprises:
a photocurable polymer formed in a structure selected from the group consisting of a cylinder, a hexahedron, an ellipsoid, a polyhedron, a circular cone, and a sphere.

15. The system of claim 13, wherein burr members of the plurality of burr members include ball terminal structures at a distal end thereof configured for minimizing potential for damage to tissues by the plurality of microrobots.

16. The system of claim 13, wherein each burr member of the plurality of burr members comprises:
a protuberance extending from a surface of the porous body, wherein the protuberance is formed in a shape selected from the group consisting of a cylinder, a hexahedron, an ellipsoid, and a polyhedron.

17. The system of claim 13, wherein the plurality of burr members are configured for culturing cells between adjacent burr members of the plurality of burr members.

18. The system of claim 13, wherein pores of the porous body are sized to facilitate supply of nutrients for tissue vascularization, and wherein adjacent burr members of the plurality of burr members of a microrobot of the plurality of microrobots have an interspacing corresponding with a type of cells to be carried by the microrobot.

19. The system of claim 13, wherein pores of the porous body of the microrobot of the plurality of microrobots are sized in correspondence with the type of cells to be carried by the microrobot, wherein the interspacing of the adjacent burr members is defined by a respective pore sized in correspondence with the type of cells to be carried by the microrobot.

20. The system of claim 13, wherein the magnetic layer comprises a metal selected from the group consisting of nickel (Ni), iron (Fe), cobalt (Co), neodymium (Nd), and combinations thereof, and wherein the biocompatible layer comprises a metal selected from the group consisting of titanium (Ti), medical stainless steel, alumina (Al₂0₃), gold (Au), and combinations thereof.

21. A method for fabricating a microrobot configured to be magnetically driven and biocompatible, the method comprising:
providing a porous body having a three-dimensional structure with a plurality of burr members, wherein the plurality of burr members are configured for carrying cells to desired sites by the microrobot between adjacent burr members of the plurality of members;
coating at least a portion of the porous body with a magnetic layer, wherein the magnetic layer is configured to enable the microrobot to be positioned at a target site using a magnetic field; and
coating at least a portion of the burr members with a biocompatible layer for cell adhesion between adjacent burr members of the plurality of burr members, wherein the biocompatible layer is configured for microrobot biostability and biocompatibility.

22. The method of claim 21, wherein the providing the porous body comprises:
using a lithographic process with a photocurable polymer to form the porous body.

23. The method of claim 21, wherein the coating the at least a portion of the porous body with a magnetic layer with the magnetic layer comprises disposing a metal on the at least a portion of the porous body selected from the group consisting of nickel (Ni), iron (Fe), cobalt (Co), neodymium (Nd), and combinations thereof.

24. The method of claim 21, wherein the coating the at least a portion of the burr members with the biocompatible layer comprises disposing a metal on the at least a portion of the burr members selected from the group consisting of titanium (Ti), medical stainless steel, alumina (Al₂0₃), gold (Au), and combinations thereof.

25. The method of claim 21, wherein the plurality of burr members are configured for culturing cells between adjacent burr members of the plurality of burr members.

26. The method of claim 25, wherein the providing the plurality of burr members comprises:
using a lithographic process with a photocurable polymer to form both the porous body and the plurality of burr members.

27. The method of claim 25, further comprising:
cultivating cells on the microrobot between adjacent burr members of the plurality of burr members.
